(19) United States
(12) Patent Application Publication
(10) Pub. No.: US 2010/0197614 A1
(43) Pub. Date: Aug. 5, 2010

(54) TREATMENT OF URINARY TRACT INFECTIONS WITH A MIXTURE OF SAPONIN AND AN ANTIBIOTIC

(76) Inventor: Soeren Kristiansen, Alleroed (DK)

Correspondence Address: JACOBSON HOLMAN PLLC, 400 SEVENTH STREET N.W., SUITE 600, WASHINGTON, DC, 20004, US

(21) Appl. No.: 12/452,327

(22) PCT Filed: Jun. 23, 2008
(86) PCT No.: PCT/DK2008/050159

§ 371 Date: Mar. 17, 2010

Related U.S. Application Data
(60) Provisional application No. 60/947,302, filed on Jun. 29, 2007.

(30) Foreign Application Priority Data
Jun. 27, 2007 (DK) PA 2007 00933

(51) Int. Cl. A61K 31/706; A61M 25/00; A61M 27/00; A61P 13/02

(52) U.S. Cl. 514/26; 604/523; 604/517

(57) ABSTRACT

Device for treating urinary infections comprising a catheter and a container, wherein the container comprises a cholesterol-binding agent. Patients who are using a urinary catheter to empty the bladder of urine are very prone to get an infection and the risk of getting a catheter associated Urinary Tract Infections (UTI) is high. The introduction of a cholesterol-binding agent facilitates a more effective curing of the infection.

### FIELD OF THE INVENTION

The present invention discloses examples of curing bladder cells for intracellular bacteria and the use of Escherichia Coli as a representative test bacteria. The invention is not limited to treat recurrent urinary tract infection (hereafter often referred to as recurrent UTI) caused by E Coli alone, but may comprise all types of microorganisms such as bacteria, fungi or vira.

### BACKGROUND

The urinary tract is one of the major sites for microbial colonisation. UTI is the most common bacterial infection of any organ. Its magnitude can be seen by the number of visits to the physicians, which is estimated to approximately 8 millions a year in the United States. In addition, patients who are using a urinary catheter to empty the bladder of urine are also very prone to get an infection and the risk of getting a catheter associated UTI is about 1-5% per catheter day (Wazait et al. 2003). Escherichia coli is the most common UTI pathogen and are involved in 40-80% of these UTIs. Other types of bacteria do also cause UTI and the UTI may be caused by one or several bacterial combinations. Under normal healthy circumstances there is no or very few bacteria in the urine, but during UTI the concentration of bacteria cell per ml urine is substantially increased. Oral intake of antibiotic(s) is the traditional way of treating the UTI and to get rid of the bacteria in the urine. The antibiotics are taken over a period of several days until the urine is free of bacteria and/or no symptoms are evident. The clinical symptoms may vary from patient to patient and the choice of antibiotic treatment may also vary from country to country and even from physician to physician. However, despite a reduction or a lack of living bacteria in the urine after the antibiotic treatment, many healthy people and catheter users experience another UTI over the succeeding days, weeks or months. This phenomenon is called recurrent UTI.

Kensil et al. U.S. Pat. No. 5,273,965 discloses methods for enhancing drug delivery with modified saponins.

### SUMMARY OF THE INVENTION

The present application starts by confirming previous findings that the test bacteria E Coli cannot survive being directly exposed for 2 hours for 100.0 μg/ml gentamicin and that bladder cells take up bacteria, illustrated by the fact that these bacteria were able to survive the antibiotic treatment. There was a close dose-response between the amount of added bacteria and the number of surviving bacteria. In other words, if more bacteria are added to the bladder cells, more bacteria are taken up and stored inside the bladder cell. The number of bacteria taken up was not higher after 2 hours compared to one hour meaning that one hour was sufficient time to take up bacteria. Now, combining saponin and the antibiotic resulted in transfer of the antibiotic inside the bladder cell and into the specialised compartment containing the bacteria where the antibiotic killed the bacteria. A combined treatment with saponin and gentamicin caused a saponin-dose-dependent kill of intracellular E Coli in irritated exfoliating bladder cells and also in bladder cells still adhering to the cell culture flask.

### DETAILED DESCRIPTION OF THE PRESENT INVENTION

The invention relates to the use of a cholesterol-binding agent—saponin(s)—that enable antibiotics and/or antimicrobial agents to get specific access to the intracellular membrane compartment harbouring the bacteria. The drug then inhibits bacterial growth or kills the bacteria inside the bladder cell. The saponin alone also irritates the bladder cells and induces cell loosening, -opening, -death, -apoptosis (programmed cell death), which all lead to exfoliation of intracellular bacteria. The combined effects reduce or clear the bladder for bacteria. The saponin alone or in combination with one or several antibiotic and/or antimicrobial agents is deposited in the bladder via a medical device or the antibiotic is administrated via intravenous or oral route.

A broad aspect of the invention relates to a method for treating a urinary tract infection comprising the step of placing a cholesterol-binding agent within the urinary bladder along with administration of an antibiotic to the patient.

The cause of recurrent UTI may be due to surviving bacteria located intracellularly in the human bladder cell.

E Coli, the most common bacteria in UTI, encodes a number of virulence factors that allows E Coli to adhere to the urinary bladder cells (see FIG. 1). The adherence is mediated by the expression of specific adhesions on the bacterial surface. The expression of adhesion on the surface of the bacteria enables the bacteria to adhere to the bladder wall and the bacteria can improve its chance to stay in the bladder and thus avoid being washed out by the normal emptying of the bladder. Many types of adhesive organelles have been characterised and are distributed among uropathogenic E Coli (Klemm and Schembri 2000). One example of adhesive organelles is the type 1 pili. The type 1 pili binds mannose-containing glycoprotein receptors on the surface of the bladder cells. Thus, mannose can substitute the mannose on the receptor and can therefore inhibit the binding of the bacterial type 1 pili to the mannose-containing receptor (see FIG. 1). The bacteria cannot adhere to the bladder cells and are washed out by the emptying of the bladder. One typical example of fighting UTI is the oral intake of cranberry juice. The juice contains specific sugar molecules that inhibit binding of E Coli bacteria to the bladder wall and the bacteria are washed out of the bladder. More important, binding of the bacteria to the bladder wall via adhesion molecules results—by an unknown mechanism—in the human bladder cell taking up some of the bacteria (Anderson et al. 2003; Bishop et al. 2007; Bower et al. 2005; Mulvey et al. 2000). This points out why recurrent UTI occurs and why the antibiotic cure is not 100% effective against the UTI. It relies on the fact that the bacteria inside the human bladder are notexposed to the antibiotic and survive the antibiotic treatment. Secondly, the bacteria are stored temporarily inside the bladder cells (Bishop et al. 2007). Later—after ending the antibiotic treatment—the bacteria are again released into the bladder urine and start another UTI episode e.g. given the phases of recurrent UTI.

The internalised bacteria are not freely distributed inside the bladder, but located in distinct membrane compartments called secretory lysosomes (Bishop et al. 2007). The membrane of secretory lysosome can fuse with the apical membrane surface the membrane facing the urine—of the bladder cell and thereby release the bacteria back into the urine (not shown in FIG. 1). Therefore, the antibiotic must migrate across 2 different types of membranes (surface/apical membrane and the lysosomal membrane) in order to get access to the intracellular bacteria. This is not possible for any type of antibiotic used to treat UTI, since many of these antibiotics are relatively highly water-soluble. Furthermore, each type of membrane is equipped with a specific content of protein and different lipid composition making antibiotic transfer impossible (see FIG. 1, panel I). In particular, the receptor for the bacteria adhesion molecule type 1 pili is the human uroplakin receptor located on the apical site of the bladder cells (Zhou et al., 2001). Uroplakin rich domains on the surface of the bladder cells are highly specific ordered regions of the plasma membrane enriched cholesterol and other lipid material and are therefore different from other membrane areas (Duncan et al., 2004). The cholesterol binds the saponin and reduces the free saponin concentration required for permeabilization of the intracellular lysosome membrane compartment. Furthermore, the antibiotic also requires retention and lack of inactivation in the final proper membrane location, which is described to be secretory lysosomes. In particular, the lysosomal pH is very low in order to activate lysosomal enzymes and the low pH value may inactivate the antibiotic. Thus, several technical obstacles have to be overcome in order to kill intracellular bacteria in the bladder cells with an antibiotic and reduce the likelihood of getting another UTI (recurring UTI). The described complex mechanism is illustratively depicted in FIG. 1, where panel I shows intracellular bacteria not accessible for the antimicrobial agent; panel II shows saponin mediated transfer of the antimicrobial agent into the secretory lysosomes and killing of the bacteria and panel III shows saponin mediated exfoliation of intracellular bacteria into the urine, where the bacteria either are killed by the antibiotic or flushed out by the urine.

Recurrent UTI is caused by localisation of hidden bacteria inside urinary bladder cells. Traditional oral administration of antibiotics cannot get access to these intracellular located bacteria and the intracellular reservoir of bacteria can later induce another UTI. The present invention uses saponin to mediate transfer of an antibacterial agent or antibiotic across the membrane barrier and into a specific membrane storage compartment containing the hidden bacteria in a proper concentration and in an active state. In combination with this, the saponin also irritates the urinary bladder cells, which concomitantly leads to bladder cell secretion (so-called exfoliation) from the bladder wall. This secretion and/or rejection of irritated bladder cells containing bacteria allow the bladder to void bacteria via the normal emptying of the bladder of urine. The combined effect of killing intracellular bacteria and exfoliation of bladder cells containing bacteria reduces the number of bacteria in the bladder wall and prevents recurrent urinary tract infections. Consequently, the present invention is particularly useful when the urinary tract infection is a recurrent urinary tract infection.

In a preferred embodiment, the cholesterol-binding agent is saponin. Several hundred different saponins can be purified from natural sources. The present preferred embodiment of saponin comprises saponin from the Quillaja bark, but can be any saponin compound. Saponins are glycosidic compounds, which comprise an aglycone compound and a saccharide compound linked together by a glycosidic bond. The aglycone part or non-saccharide part of the saponin molecule is called the genin or sapogenin. Depending on the type of genin present, the saponins according to the invention can be divided into three major classes: 1) triterpene glycosides 2) steroid glycosides and 3) steroid alkaloid glycosides. FIG. 2 illustrates the carbon skeleton of the 3 main classes of saponins according to the invention. Saponin as used herein denotes either a substantially purified saponin or one or more saponins comprised in a crude composition or a composition obtained by predetermined purification means. Saponin shall also denote any biologically active fragment of any saponin. The saponins pertaining to the present invention may be naturally occurring or synthetic or they may be made by chemical synthesis, or enzymatic synthesis involving one or more enzyme catalysed steps, either in vitro or in vivo. The preferred concentration of saponin is in the range of 0.1 to 100 micrograms/ml, but may also be in the range of 100-500 micrograms/ml. However, the optimal concentration is related to the purity of the saponin.

In one embodiment of the invention, the placement of the cholesterol-binding agent is through a catheter. Typically, the catheter used to drain urine will be used to administer the saponin. Thus, in one embodiment the catheter is a permanent e.g. a Foley-type catheter. In another embodiment of the invention, the catheter is an intermittent catheter. In one embodiment of the invention, the placement is through the urethra, for instance through a urinary catheter. In another embodiment of the invention, the placement is through a suprapubic catheter. The cholesterol-binding agent is preferably placed in the bladder. However, placement in the urethra is also conceivable.

As disclosed herein, saponin alone has beneficial effects on UTI. To get even better results, it is preferred to use a combination of saponin and antibiotic treatment. In one embodiment of the invention, the administration of the antibiotic is oral. In another embodiment of the invention, the administration of the antibiotic is intravenous. In a preferred embodiment of the invention, the administration of the antibiotic is co-administration with the cholesterol-binding agent within the bladder.

For the context herein, there is no distinction between antibiotic-, antimicrobial-, antibacterial- and/or antifungal-agents. The preferred agent is any of the agents normally used to treat UTI. Thus, the preferred embodiment of antibiotics may comprise the group of amino glycosides (gentamicin, neomycin, kanamycin, tobramycin, framycetin, streptomycin, amikacin), ampicillin and amoxillin, sulphonamides (trimethoprim-sulfamethoazole), cephalosporins, groups of beta-lactams, chloramphenicols, lincosamides, macrolides, penicillin, group of quinolones, tetracyclins and nitrafuratoin/nitrofurazone, polymyxin B, mupirocin, vancomycin, but also antimicrobial agents containing one or more biguanide groups (for example chlorhexidine or PHMB), silver complexes or silver salts, hydrogenperoxide and other oxidizing agents, quaternary ammonium compounds, agents delivering chlorine, and antimicrobial peptides.

Gentamicin can treat many types of bacterial infections, particularly Gram-negative infections. However, if gentamicin is given orally, it is not effective as it is absorbed from the small intestine and then travels through the portal vein to the liver, where it is inactivated. Further, it can also be highly nephrotoxic, particularly if multiple doses accumulate over a period of treatment. The present invention allows gentamicin to be co-administered with the cholesterol-binding agent to bypass lever inactivation and avoid nephrotoxicity. Thus, in a preferred embodiment of the invention, the antibiotic is gentamicin.

Another aspect of the invention relates to a device for treating urinary infections comprising a catheter and a container, the container comprising a cholesterol-binding agent.

Yet another aspect of the invention relates to the use of saponin for the preparation of a medicament for the treatment of recurrent urinary bladder infections in a mammal.

A related aspect relates to the use of a mixture of saponin and a bacteriocidal agent for the treatment of recurrent urinary bladder infections in a mammal. In a preferred embodiment hereof, the treatment is through administration into the bladder of the mammal.

### REFERENCES

Anderson, G. G., Palermo, J. J., Schilling, J. D., Roth, R., Heuser, J. and Hultgren, S. J. (2003) Intracellular bacterial biofilm-like pods in urinary tract infections. Science 301, 105-107.
Bishop, B. L., Duncan, M. J., Song, J., Li, G., Zaas, D. and Abraham, S. N. (2007) Cyclic AMP-regulated exocytosis of Escherichia coli from infected bladder epithelial cells. Nat. Med.
Bower, J. M., Eto, D. S, and Mulvey, M. A. (2005) Covert operations of uropathogenic Escherichia coli within the urinary tract. Traffic. 6, 18-31.
Drevets, D. A. and Campbell, P. A. (1991) Macrophage phagocytosis: use of fluorescence microscopy to distinguish between extracellular and intracellular bacteria. J. Immunol. Methods. 142, 31-38.
Duncan, M. J., Li, G., Shin, J. S., Carson, J. L. and Abraham, S. N. (2004) Bacterial penetration of bladder epithelium through lipid rafts. J. Biol. Chem. 279, 18944-18951.
Klemm, P. and Schembri, M. A. (2000) Bacterial adhesins: function and structure. Int. J. Med. Microbiol. 290, 27-35.
Mulvey, M. A., Schilling, J. D., Martinez, J. J. and Hultgren, S. J. (2000) Bad bugs and beleaguered bladders: interplay between uropathogenic Escherichia coli and innate host defenses. Proc. Natl. Acad. Sci. U.S. A 97, 8829-8835.
Teng, C. H., Cai, M., Shin, S., Xie, Y., Kim, K. J., Khan, N. A., Di Cello, F. and Kim, K. S. (2005) Escherichia coli K1 RS218 interacts with human brain microvascular endothelial cells via type 1 fimbria bacteria in the fimbriated state. Infect. Immun. 73, 2923-2931.
Wazait, H. D., Patel, H. R., Veer, V., Kelsey, M., Van Der Meulen, J. H., Miller, R. A. and Emberton, M. (2003) Catheter-associated urinary tract infections: prevalence of uropathogens and pattern of antimicrobial resistance in a UK hospital (1996-2001). BJU. Int. 91, 806-809.
Zhou, G., Mo, W. J., Sebbel, P., Min, G., Neubert, T. A., Glockshuber, R., Wu, X. R., Sun, T. T. and Kong, X. P. (2001) Uroplakin la is the urothelial receptor for uropathogenic Escherichia coli: evidence from in vitro FimH binding. J Cell Sci. 114, 4095-4103.

### FIGURES

FIG. 1, where panel I shows intracellular bacteria not accessible for the antimicrobial agent; panel II shows saponin mediated transfer of the antimicrobial agent into the secretory lysosomes and kill of the bacteria and panel III shows saponin mediated exfoliation of intracellular bacteria into the urine, where the bacteria either are killed by the antibiotic or flushed out by the urine.

FIG. 2 illustrates the carbon skeleton of the 3 main classes of saponins according to the invention.

FIG. 3, upper panel, the number of bacterial cell is reduced at very low concentrations of gentamicin showing that gentamicin inhibits the growth very effectively and also kills the bacteria at high concentrations. Furthermore, a sample of each bacteria solution at each different gentamicin concentration was transferred to an agar plate in order to test if the bacteria could give any surviving colony forming units (CFU) on an agar plate. The agar plate incubated overnight at 37° C.

The x-axis shows gentamicin concentration (μg/ml) and the y-axis shows the number of bacteria (%).

FIG. 3, lower panel, no E Coli bacteria could survive being exposed for 2 hours to concentrations of gentamicin higher than approximately 3.1 μg/ml.

FIG. 4, number of surviving bacteria on the plate expressed relatively to the number of bacteria previously added to the human bladder culture.

The x-axis shows added volume containing E Coli and the y-axis shows surviving versus added bacteria. -▪- is one hour and -▴- is two hours.

FIG. 5, green stained bacteria were located inside the bladder cells (enclosed by a white line), whereas yellow/red stained bacteria were found outside human cells as well as adjacent to the human bladder cells.

FIG. 6, upper panel, positive correlation between the expression of type 1 pili and the degree of invasion of E Coli into bladder cells. Furthermore, 50 mmol/1 mannose or glucose was added to the human bladder cells.

The x-axis shows type 1 expression (%) and the y-axis shows infection rate (%).

FIG. 6, lower panel, mannose—but not glucose—did inhibit the uptake of bacteria by the human bladder cells. The mannose interferes with the binding of the type 1 pili to the uroplakin receptor located on the entry site of the bladder cells.

The x-axis shows inhibitor and the y-axis shows infection (% of control).

FIG. 7: 50.0 micrograms/ml saponin inhibited the infection and lowering the concentration of saponin allowed the bacteria to infect the bladder cells. Fifty micrograms of saponin added after invasion and together with gentamicin resulted in total lack of detection of living bacterial cells (labelled post infection).

The x-axis shows dose of saponin and the y-axis shows infection.

FIG. 8: Permeability in HTB-9 cells saponin exposed for 2 hours. This figure shows that an increase in the concentration of saponin irritated the bladder cells with a concomitant reduction in the bladder cell number (squares). Furthermore, the reduction in the number of bladder cells was paralleled by a saponin dose-depending increase in permeability (triangles).

The x-axis shows saponin (micrograms(ml), the left y-axis shows permeability (% of control) and the right y-axis shows number of bladder cells (arbitrary units). [symbol] is permeability and [symbol] is number of bladder cells.

FIG. 9: Intracellular E Coli cells surviving gentamicin exposure (2 h, 100 ug/ml). This figure depicts the number of surviving E Coli cells in the wash (filled rectangles) containing rejected bladder cells and the number of surviving E Coli inside human bladder cells still attached to the bottom of the well (open rectangles). A combined treatment with saponin and gentamicin caused a saponin-dose-dependent kill of intracellular E Coli in irritated exfoliating bladder cells (wash, filled rectangles) and also in bladder cells still attaching to the cell culture flask (HTB 9, open rectangles).

FIG. 10 shows an example of a device according to the invention comprising a urinary catheter (10) that can be connected to a container (20) such that the mixture (30) in the container can be transferred to the bladder.

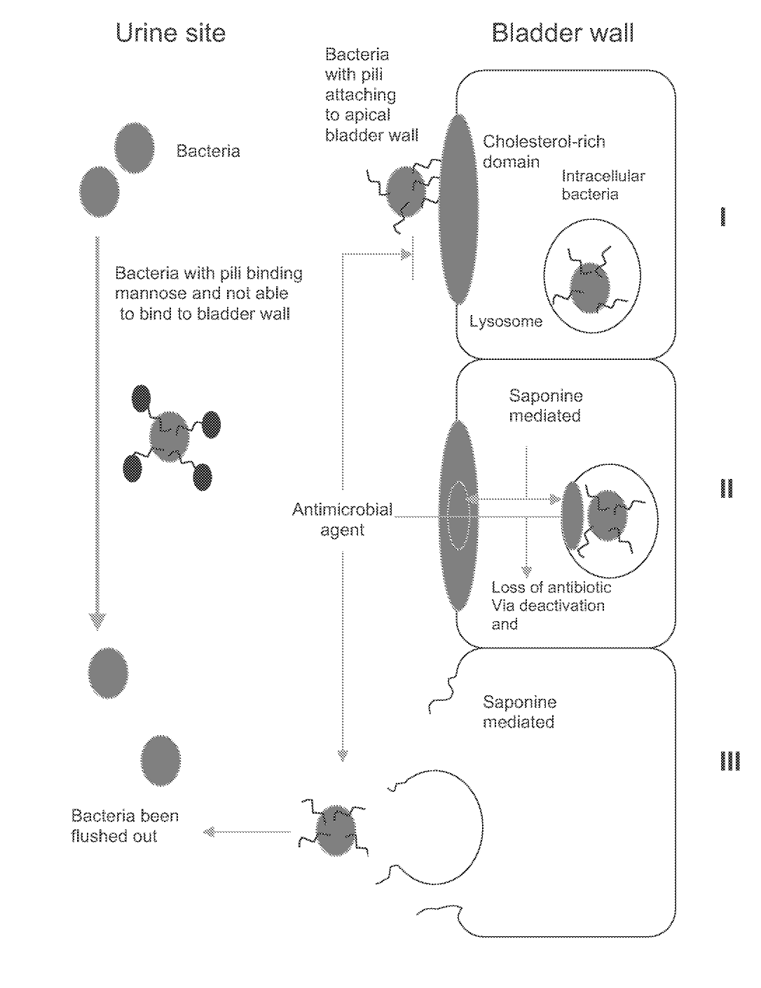

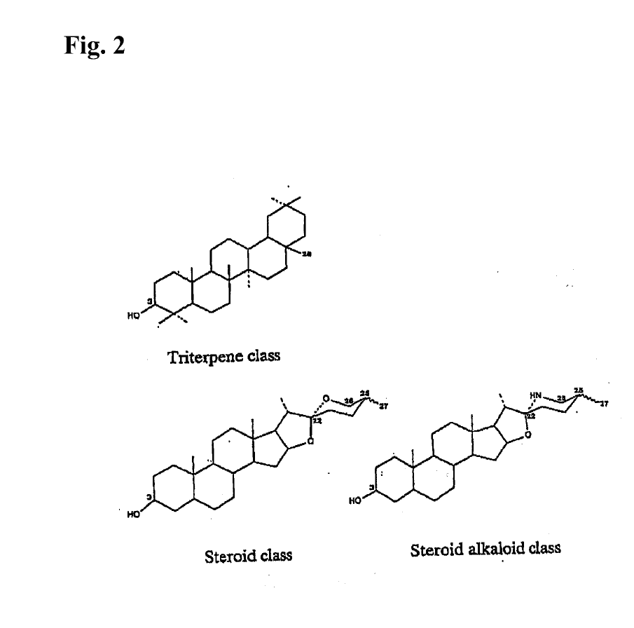

### EXAMPLES

Examples 1-4 disclose methods for measuring the number of surviving intracellularly located E Coli. The data obtained in examples 5-7 disclose the invention of using saponin to kill intracellular bacteria and exfoliation of bladder cells.

### Example 1

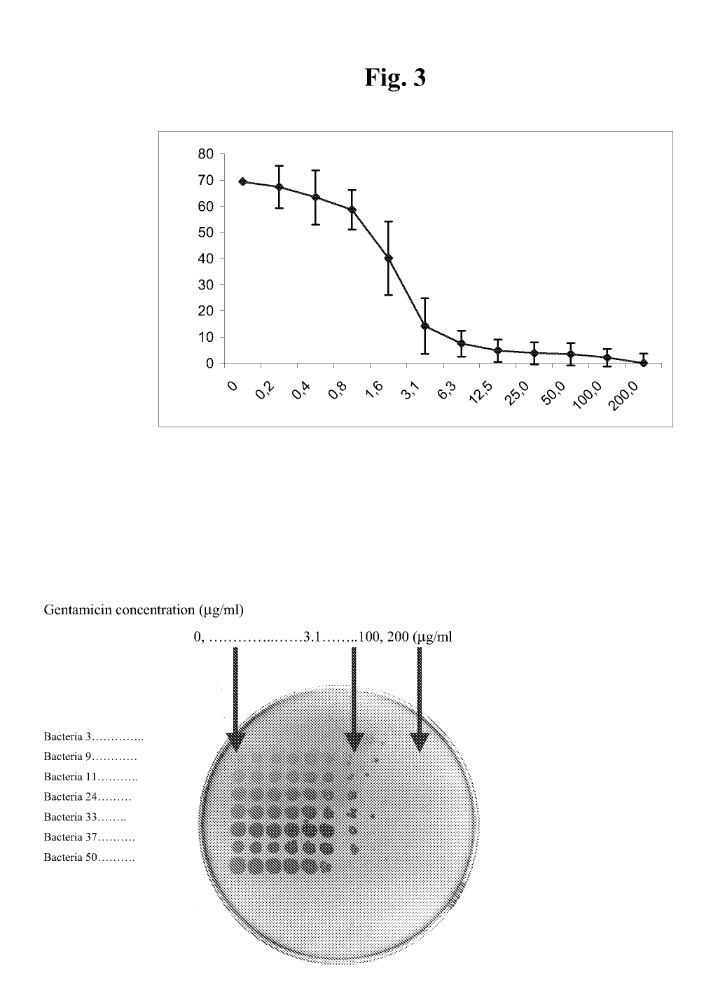

Seven different wild-type E Coli bacteria were isolated from UTI patients and were cultured in Luria Broth (LB) medium under static conditions for 2 days. The method for culturing bacteria is familiar for any skilled person in this field of microbiology. The 2 days old bacteria were diluted 100 fold in fresh sterile LB and added different concentrations of gentamicin or no gentamicin (control). Gentamicin was used as representative antibiotic agent. The bacteria were then allowed to re-grow for 2 hours under optimal growth conditions (shaking and at 37° C.) before the actual concentrations of bacteria cell in the LB were measured by determination of the absorption at 600 nm. The absorption of light reflects the number of bacteria. The number of bacteria was expressed relatively to the control preparation without gentamicin. As depicted in FIG. 3, upper panel, the number of bacterial cells is reduced at very low concentrations of gentamicin, showing that gentamicin inhibits the growth very effectively and also kills the bacteria at high concentrations. Furthermore, a sample of each bacteria solution at each different gentamicin concentration was transferred to an agar plate in order to test if the bacteria survived and could cause formation of colony forming units (CFU) on an agar plate. The agar plate incubated overnight at 37° C. As shown in FIG. 3, lower panel, no E Coli bacteria could survive being exposed for 2 hours for concentrations of gentamicin higher than approximately 3.1 μg/ml. Thus, example 1 shows that the test bacteria could not survive being directly exposed for 2 hours for 100.0 μg/ml gentamicin.

### Example 2

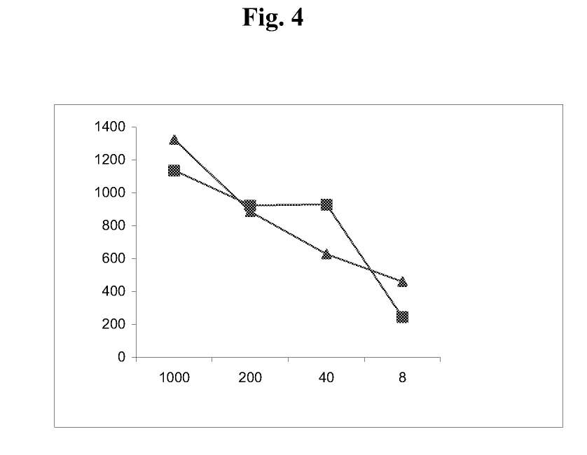

Human bladder cells were cultured and thereafter infected with bacteria. In brief, human urinary bladder cells (HTB-9 from American tissue culture collection) were cultured in culture medium and CO₂ incubator at 37° C. until the actual experiment took place. Culturing of human cells is familiar for any skilled person in this field of biology. Next, different concentrations of E Coli were added to the human bladder cells. The bladder cells and the bacteria were allowed to incubate for 1 or 2 hours together before most of the loosely bound and extracellular E Coli cells were removed by washing the bladder cells with PBS (the human bladder cells are attached to the bottom of the cell culture flask). The human bladder cells were exposed to 100.0 μg/ml gentamicin for 2 hours. Gentamicin cannot pass any membrane under normal conditions. As already shown in example 1, this gentamicin treatment kills all bacteria if the gentamicin can get access to the bacteria. Residual gentamicin and dead bacteria were removed again by washing the bladder cells with PBS. A final concentration of 0.1% triton x-100 in phosphate buffered saline was added to destroy all membrane structures of the human cells. The mixture was transferred to an agar plate and the plate was incubated overnight at 37° C. to test for surviving bacteria not having been exposed to the gentamicin. The plates contained colony-forming units as a reflection of surviving bacteria. Thus, this example shows that the bladder cell took up bacteria, since these bacteria were able to survive the antibiotic treatment. The number of surviving bacteria on the plate was expressed relatively to the number of bacteria prior added to the human bladder culture (FIG. 4). There was a close dose-response between the amount of added bacteria and the number of surviving bacteria. In other words, if more bacteria are added to the bladder cells, more bacteria are taken up and stored inside the bladder cell. The number of bacteria taken up was not higher after 2 hours compared to one hour meaning that one hour was sufficiently time to take up bacteria.

### Example 3

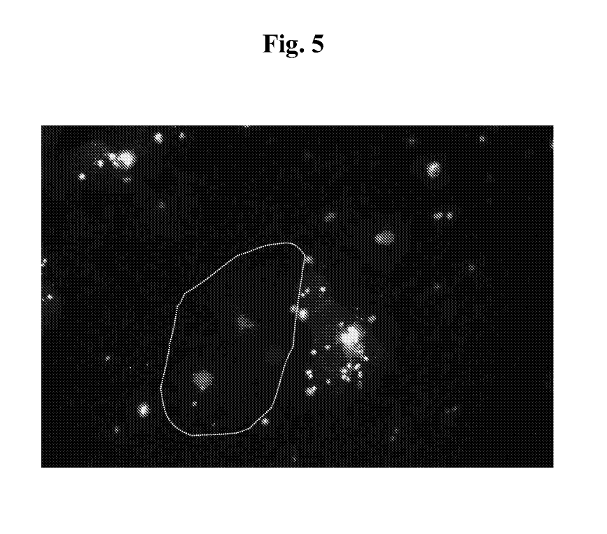

Green stained bacteria were allowed to infect human bladder cells as previous described in example 2. The infected bladder cells were then treated with ethidium bromide, as explained in detail by Drevets and Campbell 1991. The ethidium bromide turns the green stained bacteria yellow/red, if the ethdium bromide can get access to the bacteria. This experimental approach allows visual microscopic inspection of intracellular non-ethidium-bromide stained green bacteria versus extracellular red/yellow stained bacteria. As shown in FIG. 5, only green stained bacteria were located inside the bladder cells (enclosed by a white line), whereas yellow/red stained bacteria were found outside human cells as well as adjacent to the human bladder cells.

### Example 4

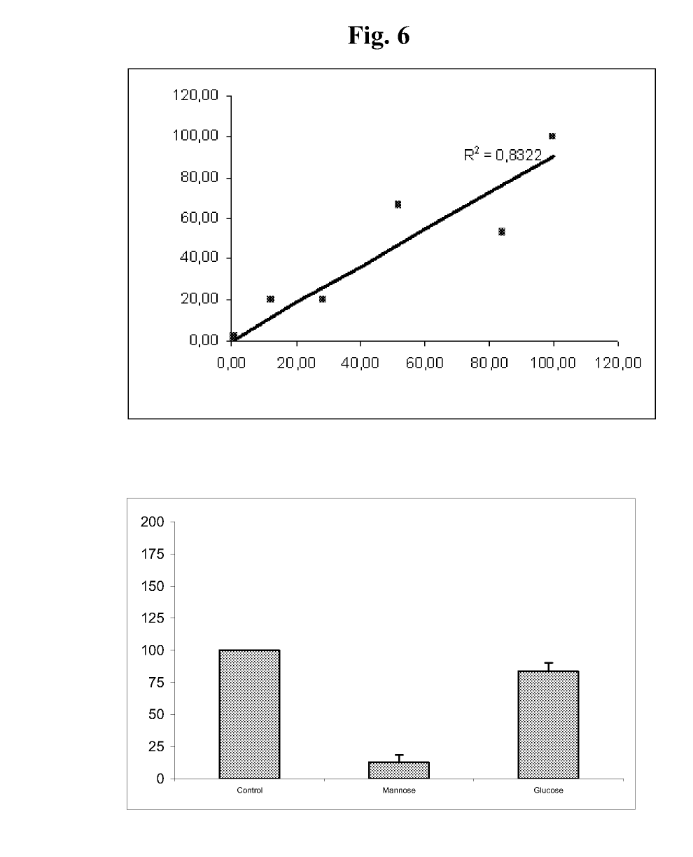

The expression of adhesive organelles—like type 1 pili—on the surface of the bacteria allows the bacteria to adhere to the bladder cells. The degree of expression of type 1 pili for the different E Coli was examined by PCR (Teng et al. 2005). PCR is a method for synthesising DNA and can be used to determine the degree of type 1 pili expression. The degree of expression of bacterial type 1 pili was correlated with the degree of bladder infection as measured by the method explained in example 2. As shown in FIG. 6 upper panel, there was a positive correlation between the expression of type 1 pili and the degree of invasion of E Coli into bladder cells. Furthermore, 50 mmol/1 mannose or glucose was added to the human bladder cells. As shown in FIG. 6, lower panel, mannose—but not glucose—did inhibit the uptake of bacteria by the human bladder cells. The mannose interferes with the binding of the type 1 pili to the uroplakin receptor located on the entry site of the bladder cells.

### Example 5

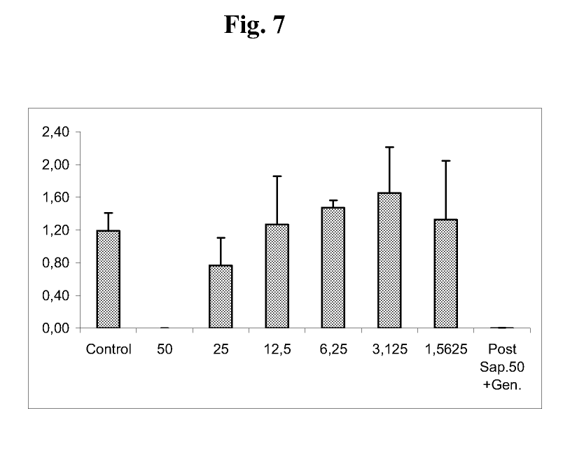

Different concentrations of saponin (ranging from 1.5 to 50 μg/ml final concentration) were added to Human bladder cells and thereafter infected with bacteria. More important, saponin in combination with 100 micrograms/ml of gentamicin (post infection) was also added to the already infected human bladder cells. As shown in FIG. 7, 50.0 micrograms/ml saponin inhibited the infection. At lower concentrations of saponin, the bacteria were allowed to infect the bladder cells. Fifty micrograms of saponin added after invasion and together with gentamicin resulted in total lack of detection of living bacterial cells (FIG. 7, labelled post infection). Thus, combining saponin and the antibiotic resulted in transfer of the antibiotic inside the bladder cell and into the specialised compartment containing the bacteria, where the antibiotic killed the bacteria.

### Example 6

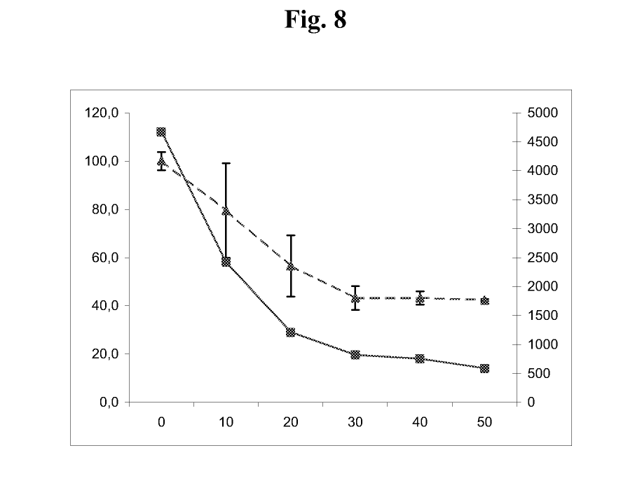

Saponin in dose dependent fashion as in example 5 was added to the human bladder cells and incubated for 2 hours. The bladder cells were washed with buffer and 2 different cell dyes; membrane permeable green SYTO 9 and membrane-impermeable red propidium iodide were added. The green stain was used to measure the number of bladder cells remaining after the dose-dependent saponin exposure and the ratio of the green/red was used to measure the degree of permeability. As shown in FIG. 8, an increase in the concentration of saponin irritated the bladder cells with a concomitant reduction in bladder cell number (green in the legend). Furthermore, the reduction in the number of bladder cells was paralleled by a saponin dose-depending increase in permeability (green/red in the legend).

### Example 7

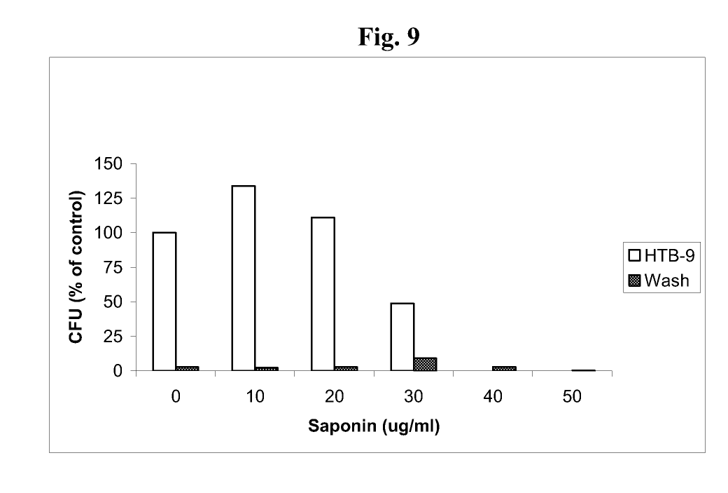

As shown in example 5, saponin enables transfer of gentamicin into the membrane compartment containing the intracellular E Coli and also initiates release of some of the bladder cells (example 6). In this example, we provide evidence that saponin and gentamicin, which were released into the medium (or urine in the bladder) also killed E Coli located inside bladder cells. In brief, the bladder cells were infected for 2 hours and then treated with saponin and gentamicin for additionally 2 hours. The number of surviving E Coli inside the bladder cells was investigated by harvesting the medium containing the released bladder cells (apoptosis and rejected bladder cells) and by investigating the bladder cells still located in the bottom of the well. FIG. 9 depicts the number of surviving E Coli cells in the wash (filled rectangles) containing rejected bladder cells and the number of surviving E Coli inside human bladder cells still attached to the bottom of the well (open rectangles). A combined treatment with saponin and gentamicin caused a saponin-dose-dependent kill of intracellular E Coli in irritated exfoliating bladder cells (labelled wash in the legend and with filled rectangles) and also in bladder cells still attaching or adhering to the cell culture flask (labelled HTB 9 in the legend and with open rectangles).

### Example 8

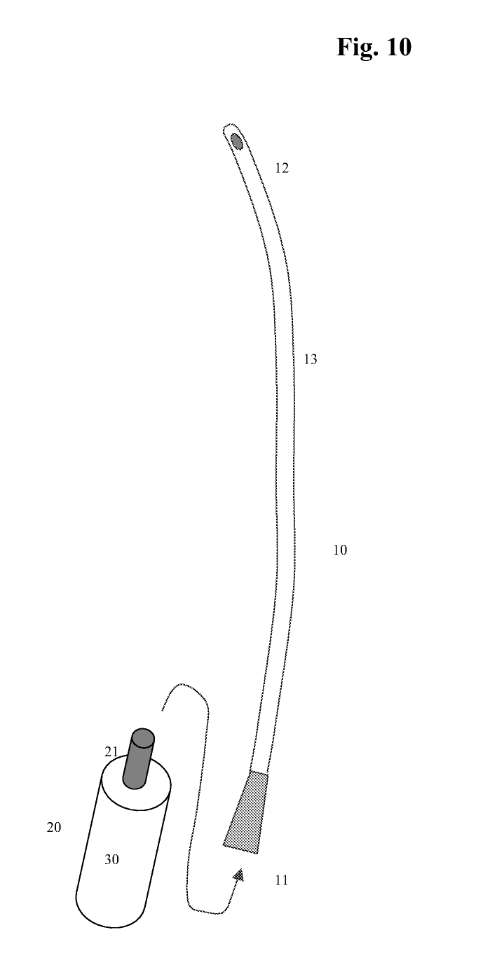

A device according to the invention is disclosed in FIG. 10. Catheter (10) comprises a distal opening (11) connected to a proximal opening (12) through a tubular part (13). The distal opening (11) can be connected to the opening (21) of the container (20). After insertion of the proximal opening (12) through the urethra (not shown) into the bladder (not shown), the bladder is emptied the usual way. Then the container opening (21) is attached to the distal catheter opening (11) and the container (20) is lifted above bladder height in a way that the mixture (30) will pass through the container opening (21) through the distal catheter opening (11), through the tubular part (13) and through the proximal opening (12) into the bladder. When the container (20) is empty, the catheter is withdrawn leaving the mixture in the bladder.

The mixture is 20 ml 0.9% NaCl, comprising saponin (50 μg/ml) and gentamicin (100.0 μg/ml).

1. Device for treating urinary infections comprising a catheter and a container, the container comprising a cholesterol-binding agent.

2. Device according to claim 2, wherein the catheter is a urinary catheter.

3. Device according to claim 1, wherein the cholesterol-binding agent is saponin.

4. Device according to claim 1, wherein the device further comprises an antibiotic for co-administration with the cholesterol-binding agent within the bladder.

5. Device according to claim 1, wherein the antibiotic is gentamicin.

6. Method for treating and/or preventing a urinary tract infection comprising the step of placing a cholesterol-binding agent within the urinary bladder.

7. Method according to claim 6, further comprising the step of administration of an antibiotic to the patient.

8. Method according to claim 6, wherein the urinary tract infection is a recurrent urinary tract infection.

9. Method according to claim 6, wherein the cholesterol-binding agent is a saponin.

10. Method according to claim 6, wherein the step of placing the cholesterol-binding agent is through a urinary catheter.

11. Method according to claim 6, wherein the administration of antibiotic is oral.

12. Method according to claim 6, wherein the administration of the antibiotic is intravenous.

13. Method according to claim 6, wherein the administration of the antibiotic is co-administration with the cholesterol-binding agent within the bladder.

14. Method according to claim 7, wherein the antibiotic is bacteriocidal.

15. Method according to claim 14, wherein the bacteriocidal antibiotic is gentamicin.

16. Use of saponin for the preparation of a medicament for the treatment of recurrent urinary bladder infections in a mammal.

17. Use of a mixture of saponin and a bacteriocidal agent for the treatment of recurrent urinary bladder infections in a mammal.

18. Use according to claim 17, wherein the treatment is through administration into the bladder of the mammal.
